(19) United States
(12) Patent Application Publication
(10) Pub. No.: US 2023/0154227 A1
(43) Pub. Date: May 18, 2023

(54) A TERAHERTZ BIOMETRIC IMAGING PACKAGE

(71) Applicant: Fingerprint Cards Anacatum IP AB, Göteborg (SE)

(72) Inventor: Johan HAMMERSBERG, FLODA (SE)

(73) Assignee: Fingerprint Cards Anacatum IP AB, Göteborg (SE)

(21) Appl. No.: 17/909,371

(22) PCT Filed: Mar. 10, 2021
(86) PCT No.: PCT/SE2021/050211

§ 371 Date: Sep. 3, 2022

(30) Foreign Application Priority Data
Mar. 13, 2020 (SE) 2050277-9

(51) Int. Cl. G06V 40/13; H04N 25/21; H04N 25/78; A61B 5/0507; H01L 27/146

(52) U.S. Cl. CPC G06V 40/13; H04N 25/21; H04N 25/78; A61B 5/0507; H01L 27/14618; H01L 27/14692; A61B 2562/046; A61B 2503/12; H01L 31/112

(57) ABSTRACT

The present invention relates to a terahertz biometric imaging package comprising: an image sensor comprising an antenna pixel array arranged to detect terahertz radiation transmitted from an object, for capturing an image, each antenna pixel comprises a power detector including an antenna structure for receiving terahertz radiation, wherein the power detector is configured to convert a detected terahertz radiation to a sensing signal at a lower frequency than the frequency of the terahertz radiation, a package top cover arranged to cover the antenna pixel array, wherein the image sensor is configured to capture a terahertz image of an object located on an opposite side of the package top cover, a package bottom part arranged on the other side of the antenna pixel array opposite from the package top cover, wherein the antenna pixel array is encapsulated between the package top cover and the package bottom part.

### FIELD OF THE INVENTION

The present invention relates to a terahertz biometric imaging package, to an electronic device, and to a method for manufacturing an image sensor for a terahertz biometric imaging package.

### BACKGROUND OF THE INVENTION

Biometric systems are widely used as means for increasing the convenience and security of personal electronic devices, such as mobile phones etc. Fingerprint sensing systems, in particular, are now included in a large proportion of all newly released consumer electronic devices, such as mobile phones.

Optical fingerprint sensors have been known for some time and may be a feasible alternative to e.g. capacitive fingerprint sensors in certain applications. Optical fingerprint sensors may for example be based on the pinhole imaging principle and/or may employ micro-channels, i.e. collimators or microlenses to focus incoming light onto an image sensor. Capacitive fingerprint sensors rely on capacitive coupling between the fingerprint features of a finger and capacitive plates of the sensor.

Generally, it is desirable to integrate fingerprint sensing systems in electronic devices or in other devices, in a manufacturing efficient and cost-efficient way.

Although both optical fingerprint sensor and capacitive sensors provide promising integration solutions, there is still room for improvements with regards to fingerprint sensing system integration.

### SUMMARY

In view of the above-mentioned and other drawbacks of the prior art, it is an object of the present invention to provide a biometric sensor based on terahertz imaging technology that is provided as a package with improved integration possibilities compared to prior art fingerprint sensing systems.

According to a first aspect of the invention, there is provided a terahertz biometric imaging package comprising an image sensor comprising an antenna pixel array arranged to detect terahertz radiation transmitted from the illuminated object, for capturing an image. Each antenna pixel comprises a power detector including an antenna structure for receiving terahertz radiation, wherein the power detector is configured to convert a detected terahertz radiation to a sensing signal at a lower frequency than the frequency of the terahertz radiation.

Further, the terahertz biometric imaging package comprises a package top cover arranged to cover the antenna pixel array, wherein the image sensor is configured to capture a terahertz image of an object located on an opposite side of the package top cover.

Additionally, the package comprises a package bottom part arranged on the other side of the antenna pixel array opposite from the package top cover, wherein the antenna pixel array is encapsulated between the package top cover and the package bottom part.

The present invention is at least partly based on the realization that a terahertz image sensor may provide for a compact overall imaging package that provides for integration in a vast number of applications. Compared to other sensing technologies, terahertz sensors open for new possibilities in sensor configurations both in terms of package component material and design, but also in size of the total package. For example, as will be described below, the package bottom part or top cover may themselves serve as substrate for the image sensor pixels.

The present invention is further at least partly based on the realization that imaging at millimeter and sub-millimeter wavelengths, e.g. at frequencies in the “terahertz gap”, provides for increasing the ability to detect structures under the outermost tissue layer of a fingerprint. In other words, sub-dermal layers of the fingerprint may be detected. It was realized that the wavelength in the terahertz gap is long enough to be detected using e.g. RF circuit design but also low enough to be considered as light when it comes to beam shaping optics that may be implemented in some embodiments. Further, the photon energy is low enough so that the photons are not absorbed in most materials, i.e. the penetration of the radiation may be e.g. up to 0.2 mm, into the skin, increasing the probability to detect fingerprint spoofs.

Further, by means of the claimed invention, it may be possible to detect a fingerprint without requiring direct contact between the skin and the image sensor, and this ability is improved by the penetration of the radiation, thereby providing for more integration possibilities than for e.g. optical sensors operating in the visible range of light, or capacitive sensors requiring physical contact with the sensor surface.

Terahertz is herein preferably meant to include a range of radiation frequencies that are below the frequency of infrared light and above the frequency of microwaves, e.g. range of terahertz may herein be about 100 GHz to about 10 THz.

By the provision of the antenna pixel as a power detector, a compact antenna pixel is obtained that allows for simple read-out since the signal output is already, on chip, adapted for an analogue to digital converter (ADC) to receive without requiring additional AC-to-DC conversion circuits. Thus, the antenna pixels comprise both the antenna itself for collecting the terahertz radiation, and a frequency converting element for converting the detected terahertz signal to a signal detectable by e.g. an ADC. The inventors thus realized an array of such compact power detectors for image capturing in the terahertz range for applications where space is often limited.

A power detector acts as a sensor to detect the terahertz radiation and to provide a DC signal, which level depends on the power of the detected terahertz radiation. In other words, the antenna pixel may be adapted to sense the incoming terahertz radiation and to output a low frequency signal or a DC voltage level or a DC current level that is based on the power of the detected incoming terahertz radiation. Thus, a level of a DC voltage or current output from an antenna pixel may be based on the power of the detected terahertz radiation.

Still further, a power detector may advantageously be made from a two-dimensional material. Such two-dimensional material is preferably suitable for high-frequency applications. A two-dimensional material generally only includes one or a few atom layers.

For example, the two-dimensional material may be graphene. More specifically, the antenna pixel array may be made from graphene. Graphene is an example two-dimensional material and comprises one or a few layers of carbon atoms. Further, graphene is particularly suitable for the antenna and/or the power detector since graphene has high electrical mobility which means it allows for fast operation of a transistor structure made from graphene. Such transistor may be a graphene field effect transistor. Further, the electrical properties of graphene enable for modulating the electrical conductivity in a gate of a graphene structure which advantageously enables for frequency conversion for simple read-out as described above.

Further, graphene is a two-dimensional material that is flexible or bendable when arranged on a flexible or bendable substrate which provides mounting advantages for a vast number of applications.

Contrary to bulk semiconductor transistors, graphene is a two-dimensional material and provides improved sensitivity compared to the conventional bulk transistors. For example, the gate, drain and source structures of a graphene FET transistor may serve as antenna, whereby the flow of current from source to drain is affected by terahertz radiation that impedes on the gate/antenna.

In addition, using graphene for the antenna pixel enables for an at least nearly optically transparent antenna pixel array. The advantageously allows for nearly arbitrary mounting location of the image sensor in locations where it is desirable to not visually obstruct the appearance other components.

Although graphene is an advantageous alternative for embodiments herein, other two-dimensional materials are also conceivable, such as e.g. silicene, germanene, and phosphorene but also transition metal dichalcogenides (TMDs) such as e.g., MoS₂, WS₂, WSe2.

In some possible implementations, where non-flexible and opaque image sensors are conceivable, materials such as InP and GaN may be used for creating high frequency devices such as HEMT transistor-based power detectors.

The antenna may be a dipole antenna e.g. employing a bow tie antenna configuration. A bow tie antenna typically employs an at least partly circular geometry which advantageously provides a more polarization independent antenna compared to dipole antennas employing more straight geometries. Thereby, using a bow tie antenna provides for increasing the signal strength of detected terahertz radiation.

The power detector may hereby be an on-chip transistor structure electrically connected to the antenna of the pixel. Preferably, the antenna structure is part of the transistor structure.

The transistor structure and the antenna structure may be made in a single component, i.e. as a one component power detector. Thus, the pixel itself may comprise both the antenna and the transistor for rectifying the detected signal to provide a sensing signal.

The sensing signal is extractable from the image sensor for redirecting to an analogue to digital converter of a read-out circuit.

As mentioned above, the image sensor in embodiments of the present invention provides for improved flexibility in mounting locations and selection of material and design of packaging components, i.e. the package top cover and the package bottom part.

Accordingly, in embodiments, the package bottom part may be configured as a substrate for the array of antenna pixels. For example, the array of antenna pixels may advantageously be manufactured on the package bottom part, thereby reducing the number of parts of the package and the size of the package

Analogously, in other embodiments, the package top cover may be configured as a substrate for the array of antenna pixels. For example, the array of antenna pixels may advantageously be is manufactured on the package top cover, thereby reducing the number of parts of the package and the size of the package

In cases where the image sensor comprises a substrate supporting the antenna pixel array, wherein the substrate may advantageously be made from a flexible material.

Preferably, the antenna pixel array is a two-dimensional array of antenna pixels.

In embodiments, the package top cover may be a flexible transparent film. This advantageously allows for a wide range of mounting locations on surfaces that are bent or curved, or surfaces comprises features that should be visible, e.g. using a two-dimensional material for the image sensor, the package may be transparent if the bottom cover part is also transparent.

Accordingly, in embodiments, the package bottom part may be a flexible transparent film. For example, arranging the image sensor provided as an array of pixel antennas made from a two-dimensional material achieves an optically transparent and compact biometric imaging package. Such a biometric imaging package is mountable on nearly any surface since it is flexible and transparent and can be thin mainly limited by the thickness of the flexible transparent films. For example, the biometric imaging package is directly attachable to a surface of a user device. One possible implementation is that the biometric imaging package is attached to the outer surface of a display cover glass. In other words, the display may be manufactured nearly independently of the biometric imaging package which may be mounted on the outer surface of the display, i.e. on the side of the cover glass facing the user.

To provide an easy mountable and compact biometric imaging package the package top cover and the package bottom part may be attached to each other with the array of antenna pixels in-between.

The image sensor of the biometric imaging package may be adapted to passively detect terahertz radiation produced by the object itself, without the need for assisting illumination that illuminates the object. This provides for eliminating the need for a source that is fast enough to produce sufficient power at frequencies covering the terahertz frequencies, preferably in the terahertz gap discussed above. Further, by eliminating the source, a more compact biometric imaging device is obtained which is less complicated to mount in various locations.

However, equally well, the image sensor of the biometric imaging package may be adapted to detect radiation that is reflected from the object. In such case, terahertz radiation is emitted for illuminating the object and the image sensor is arranged to detect terahertz radiation reflected off the object.

Thus, the terahertz biometric imaging package may comprise a transmitter element arranged to emit terahertz radiation for illuminating the object.

The transmitter element and the array of antenna pixels may advantageously be arranged on the same substrate. The substrate may be, as described above, the package top cover or the package bottom part.

For example, an array of transmitter elements may be arranged interleaved with the array of antenna pixels on the same substrate surface. In other words, a mixed array of antenna pixels and transmitter element may be arranged on the substrate in the same plane. This provides for a homogenous illumination of the object as seen from the antenna pixel array, thereby improving image quality.

Various types of transmitter element are conceivable. For example, the transmitter element may comprise a thermal emitting filament which may be provided as a filament blackbody radiation layer, transmitting radiation in the terahertz range. This blackbody radiation layer may be combined with reflector layer to guide the radiation toward the finger where the reflection by the finger will occur. The input power to the black body filament radiator layer can be pulse modulated to ease the noise suppression in the detector circuit, for example using lock-in techniques or similar.

In other possible embodiments, the transmitter element may comprise at least one non-linear device diode or a transistor. One example is a so-called negative resistance oscillator.

The image sensor is operative to detect terahertz radiation in a frequency range excluding the range of visible light. The visible range is understood to be for humans and is in the range of about 400 nm to 700 nm.

The image sensor thus comprises antennas that are designed to couple to the terahertz frequencies of radiation. The image sensor may be operative at frequencies in the terahertz range, e.g. 10 GHz to 100 THz. The image acquired by the image sensor may be considered a terahertz image.

The antennas are micro-sized antennas, e.g. in the range of a micrometers, to thereby fit a large number of antennas in the antenna pixel array. Further, the dimension and design of the antenna and the associated circuitry provides for tuning the antenna pixel for a specific terahertz frequency range. The size of an example antenna pixel may be in the range of about 15 micrometers to about 150 micrometer.

Preferably, the image sensor is operative in the frequency range 10 GHz to 100 THz, preferably, 100 GHz to 50 THz, more preferably 300 GHz to 30 THz.

The outer surface of the package top cover may in some implementations also be referred to as a sensing surface. The operating principle of the described biometric imaging arrangement is that radiation emitted by the transmitter element will be reflected by a finger placed on the sensing surface, and the reflected radiation is received by the antennas in the antenna pixel array which produce sensing signals indicative of the detected terahertz radiation. Alternatively, for the passive detection principle, terahertz radiation produced by the finger itself is received by the antennas. By combining the signals from all the antennas, an image representing the fingerprint can be formed and subsequent biometric verification can be performed.

According to a second aspect of the invention, there is provided an electronic device comprising the terahertz biometric imaging package according to embodiments, and processing circuitry configured to: receive a signal from the terahertz biometric imaging arrangement indicative of a biometric object touching the transparent display panel, perform a biometric authentication procedure based on the detected fingerprint.

Biometric authentication procedures such as fingerprint authentication procedures are known per se, and generally includes to compare features of a verification representation constructed based on an acquired fingerprint image, with features of an enrollment representation constructed during enrollment of a user. If a match with sufficiently high score is found, the user is successfully authenticated.

The biometric object may be a finger, whereby the signal is indicative of a fingerprint of the finger.

The electronic device is a mobile device, such as a mobile phone (e.g. Smart Phone), a tablet, a laptop, smart card, or any other portable device.

Further effects and features of the second aspect of the invention are largely analogous to those described above in connection with the first aspect of the invention.

According to a third aspect of the invention, there is provided a method of manufacturing an image sensor for a terahertz biometric imaging package, the method comprising: providing a package bottom part and a package top cover for a terahertz biometric imaging package; providing a layer of a two-dimensional material on a surface of the package bottom part or the package top cover; patterning the layer of two-dimensional material to form an array of antenna pixels configured to detect terahertz radiation.

Providing a layer of a two-dimensional material on a surface may comprise depositing the two-dimensional material on the surface. Techniques available for depositing the two-dimensional material include standard thin film technology such as e.g. chemical vapor deposition for graphene, or sputtering, pulsed laser deposition, physical vapor deposition, e-beam lithography or photolithography, etching, etc.

In embodiments, the package bottom part and the package top cover may be flexible and transparent films, whereby the method may comprise laminating the flexible and transparent films to each other such that the array of antenna pixels is enclosed therebetween.

The two-dimensional material may be deposited directly on the package bottom or top, or the two-dimensional material may be transferred from a substrate onto the package bottom or top. Other materials needed for the antenna pixels such as metal lines and dielectric materials may be deposited directly on the package bottom or top using known microfabrication techniques.

Further effects and features of the third aspect of the invention are largely analogous to those described above in connection with the first aspect and the second aspect of the invention.

Further features of, and advantages with, the present invention will become apparent when studying the appended claims and the following description. The skilled person realize that different features of the present invention may be combined to create embodiments other than those described in the following, without departing from the scope of the present invention.

### BRIEF DESCRIPTION OF THE DRAWINGS

These and other aspects of the present invention will now be described in more detail, with reference to the appended drawings showing an example embodiment of the invention, wherein:

FIG. 1 conceptually illustrates a biometric terahertz imaging package according to embodiments of the invention;

FIG. 2A conceptually illustrates an antenna pixel array and an individual antenna pixel according to an embodiment of the invention;

FIG. 2B conceptually illustrates an example antenna pixel according to an embodiment of the invention;

FIG. 3A conceptually illustrates a biometric terahertz imaging package with the antenna pixels arranged on the bottom cover part according to embodiments of the invention;

FIG. 3B conceptually illustrates a biometric terahertz imaging package with the antenna pixels arranged on the top cover according to embodiments of the invention;

FIG. 4A conceptually illustrates a side-view of a terahertz biometric imaging package according to a preferred embodiment of the invention;

FIG. 4B is a perspective view of a terahertz biometric imaging package according to a preferred embodiment of the invention;

FIG. 5 conceptually illustrates a possible implementation of the terahertz biometric imaging package according to embodiments of the invention;

FIG. 6A conceptually illustrates a possible implementation of the terahertz biometric imaging package according to embodiments of the invention;

FIG. 6B conceptually illustrates a side-view of a possible implementation of the terahertz biometric imaging package according to embodiments of the invention;

FIG. 6C conceptually illustrates a side-view of a possible implementation of the terahertz biometric imaging package according to embodiments of the invention;

FIG. 7 conceptually illustrates a biometric terahertz imaging package according to embodiments of the invention;

FIG. 8 conceptually illustrates a sensing circuitry for read-out of a sensing signal from an antenna in the antenna pixel array;

FIG. 9 conceptually illustrates a sensing circuitry for read-out of a sensing signal from an antenna in the antenna pixel array;

FIG. 10 conceptually illustrates a sensing circuitry for read-out of a sensing signal from an antenna in the antenna pixel array;

FIG. 11 conceptually illustrates a transmitter element in the form of a black body radiation element;

FIG. 12 conceptually illustrates a transmitter element in the form of a negative resistance oscillator;

FIG. 13 is a schematic box diagram of an electronic device according to embodiments of the invention; and

FIG. 14 is a flow-chart of method steps according to embodiments of the invention.

### DETAILED DESCRIPTION OF EXAMPLE EMBODIMENTS

In the present detailed description, various embodiments of the terahertz biometric imaging package according to the present invention are herein described with reference to specific implementations. However, it should be noted that the described terahertz biometric imaging package also may be used for other biometric imaging implementations.

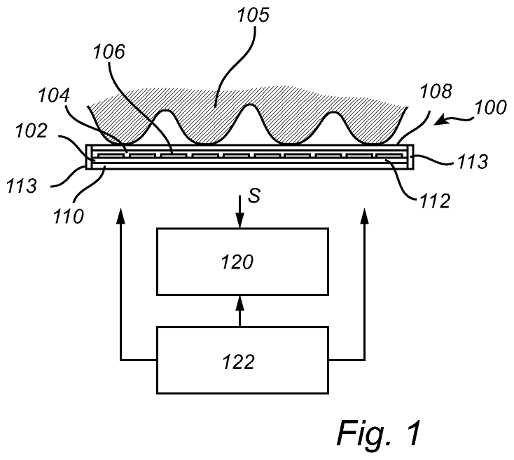

FIG. 1 conceptually illustrates a biometric terahertz imaging package 100 according to embodiments of the invention. The biometric terahertz imaging package 100 comprises an image sensor 102 comprising an antenna pixel array 104 arranged to detect terahertz radiation transmitted from an object 105, for capturing an image.

Each antenna pixel 106 comprises a power detector including an antenna structure for receiving terahertz radiation, wherein the power detector is configured to convert a detected terahertz radiation to a sensing signal at a lower frequency than the frequency of the terahertz radiation. The lower frequency may be at DC.

Further, a package top cover 108 is arranged to cover the antenna pixel array 104, wherein the image sensor is configured to capture a terahertz image of an object 105 located on an opposite side of the package top cover 108.

A package bottom part 110 is arranged on the other side of the antenna pixel array 104 opposite from the package top cover 108. In this way is the antenna pixel array 104 encapsulated between the package top cover 108 and the package bottom part 110.

The package top cover 108 and the package bottom part 110 are attached to each other with the array 104 of antenna pixels in-between.

The package 100 may include side walls 113 being separate side walls or being part of the package bottom part 110, or being part of the top cover 108, although other possibilities are conceivable as will be described herein.

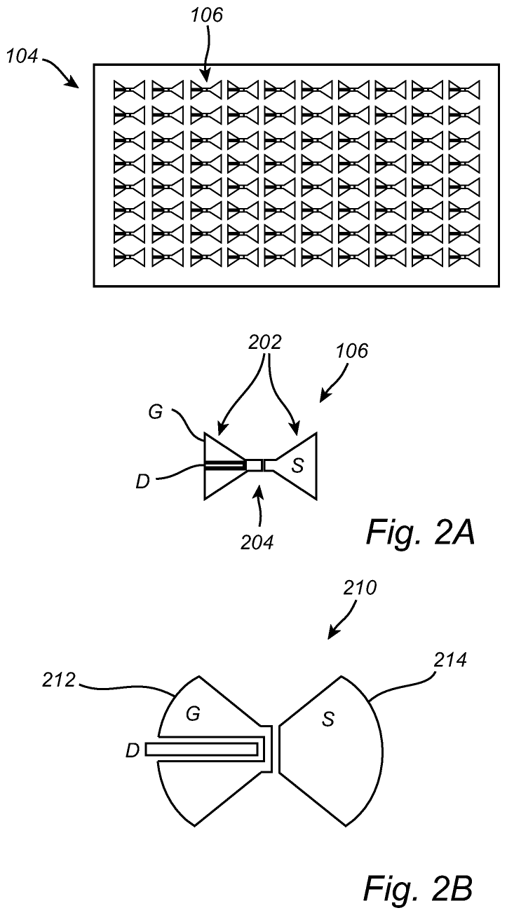

Using terahertz imaging technology enables for new possibilities in packaging and imaging performance. Firstly FIG. 2A, conceptually illustrates an example antenna pixel array 104 in the form of a two-dimensional array 104 of antenna pixels 106. Each antenna pixel 106 includes an antenna structure 202 and a transistor 204. The antenna structure 202 may be the gate G and source S of the transistor 204. In this specific example embodiment, the antenna pixel 106 is a dipole antenna sensor. The transistor 204 may be made by e.g. standard semiconductor Si, InP, InAsP, GaN, SiGe transistors or similar.

In one advantageous embodiment, the antenna structure 202 and the transistor 204 are made in a two-dimensional material, in a single layer. For example, the two-dimensional material may be graphene although other two-dimensional materials are also conceivable. In some embodiments, the transistor 204 may be a graphene field effect transistor (GFET).

The antenna pixel array 104 in this embodiment may be manufactured using standard thin film technology such as e.g. chemical vapor deposition for graphene, or sputtering, pulsed laser deposition, physical vapor deposition, e-beam lithography or photolithography, etching, etc.

The transistor and antenna together serve to detect, by the antenna, and convert, by the transistor, a detected terahertz radiation impinging on the antennas to a signal at a lower frequency than the frequency of the terahertz radiation. Advantageously, the antenna structure 202 and the transistor 204 are integrated in a single component on-chip.

The antenna pixel 106 is configured as a power detector adapted to detect the terahertz radiation and output a DC or low frequency signal related to the power of the incoming terahertz radiation. The transistor 204 serves as a rectifying element of the power detector 106. In other words, the antennas, i.e. the gate and the source, are configured to receive the terahertz radiation, and the transistor is configured to convert and rectify the received signal to a DC or low frequency signal. The DC or low frequency signal may be read by an ADC.

In other words, now turning to FIG. 1 again, the image sensor 108 is connected to an analog-to-digital converter 120 for sampling and converting the analog signals S originating directly from the antenna pixels 110 to a digital representation of the fingerprint pattern of the finger 104. Further, the image sensor 108 is connected to, as conceptually illustrated by arrows, suitable column and row control and timing circuitry 122 such as including application specific integrated circuits (ASICs) and field programmable gate arrays (FPGA), and multiplexers.

Accordingly, the sensing signal S is extractable from the image sensor 102, e.g. through suitable feedthroughs in the package top cover or package bottom part, for redirecting to an analogue to digital converter 120 of a read-out circuit.

The antenna structure 202 and the transistor structure 204 may be made in a single layer, thereby providing an antenna pixel array 104 that is relatively simple to manufacture. The antenna may be a planar antenna, thereby providing an image sensor that advantageously barely contributes to the stack-up of the biometric imaging sensor, thus providing a thin image sensor.

FIG. 2B conceptually illustrates another example antenna pixel 210, e.g. a power detector of bow tie configuration. The power detector 210 comprises a gate G, a source S, and a drain D. The geometry of the gate G and source S at least partly determines the resonance frequency that the power detector is tuned at. More precisely, the resonance frequency of the power detector, is defined by the electrical coupling between the drain D and source S and gate G, and the geometry of the various parts of the power detector. Preferably, the operative frequency range of the antenna pixel is included in the range of 10 GHz to 100 THz, preferably 100 GHz to 50 THz, more preferably 300 GHz to 30 THz.

Here, the gate G and source S of the bow-tie power detector 210 each comprises a curved distal edge 212 and 214, respectively. In other words, the gate G and source S each comprise one end that is shaped with a predetermined radius of curvature as seen from above. The shape of the distal ends 212 and 214 may be adapted for tuning the operation frequency of the power detector 210. Further, the at least partly circular geometry provided by the curved distal ends 212, 214 advantageously provides a more polarization independent antenna compared to dipole antennas employing more straight geometries.

In FIG. 1, the antenna pixels 106 are arranged on a separate substrate 112 that is attached to the cover bottom part 110. This is one of many possible implementations. In preferred embodiments, the substrate is omitted, and the array of antenna pixels is manufactured directly on the package bottom part or directly on the package top cover. Thus, the package top cover 108 or the package bottom part 110 may serve as a substrate for the antenna pixels 106, 210.

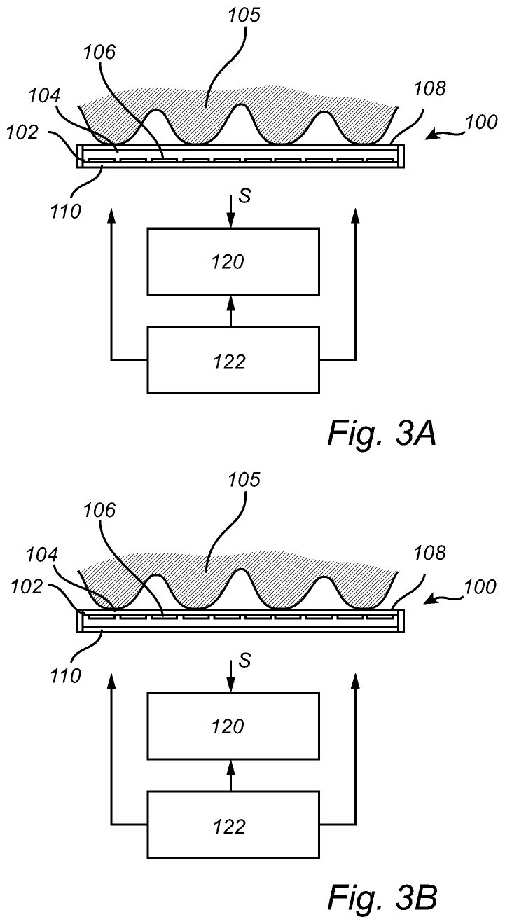

FIG. 3A illustrates the antenna pixels 106 being arranged on the bottom cover part 110 serving as a substrate for the antenna pixels 104.

FIG. 3B illustrates the antenna pixels 106 being arranged on the top cover 108 serving as a substrate for the antenna pixels 104.

The embodiments shown in FIGS. 3A-B provides for a compact terahertz biometric imaging package 100 where the dedicated substrate for the antenna pixels may be omitted. The manufacturing of the terahertz biometric imaging package thus includes processing steps for manufacturing antenna pixels, and signal routing lines, directly on other parts of the package, i.e. the bottom cover part 110 or the top cover 108. For example, the bottom cover part 110 or the top cover 108 may be made from a flexible material which allows for, when selecting a suitable material for the antenna pixel array, arrangement of the terahertz biometric imaging package on curved surfaces.

Manufacturing the power detectors from a two-dimensional material, such as graphene, advantageously enables for providing a flexible image sensor if the substrate for the power detector is a flexible substrate. The flexible substrate may comprise of e.g. PET (Polyethylene terephthalate), PEN (Polyethylene naphthalate), or any other similar materials. In embodiments, the top package cover or the bottom cover part is adapted as a substrate for the power detectors.

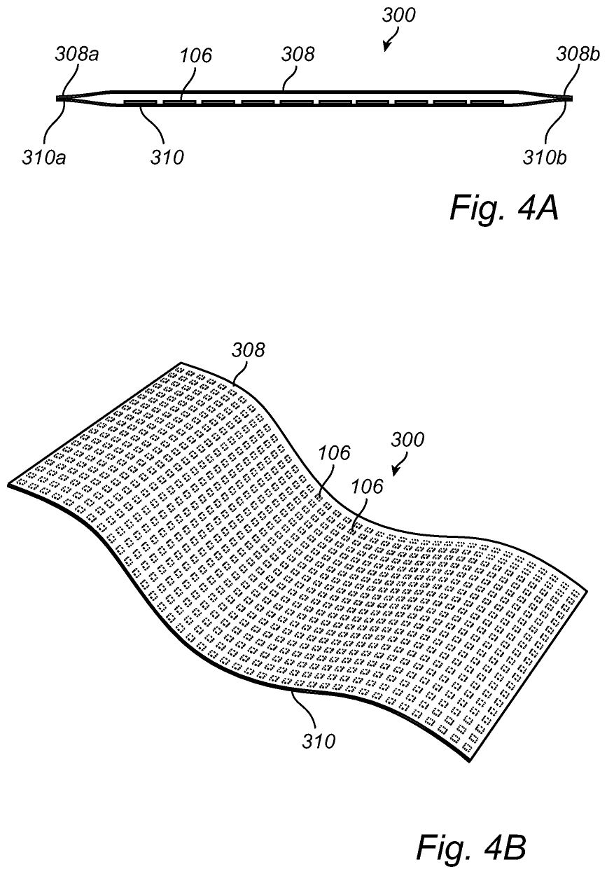

FIG. 4A conceptually illustrates a terahertz biometric imaging package 300 according to a further preferred embodiment of the invention. In this embodiment, the package top cover is a flexible transparent film 308. Further, the package bottom part is a flexible transparent film 310. The array of antenna pixels 106 is arranged sandwiched between the top flexible film 308 and the bottom flexible film 310. The top flexible film 308 and the bottom flexible film 310 are attached to each other at their edge portions to thereby fully enclose the antenna pixels 106 between them. More precisely, the first edge portion 308a of the top flexible film 308 is attached to the first edge portion 310a of the bottom flexible film, and the second edge portion 308b of the top flexible film 308 is attached to the second edge portion 310b of the bottom flexible film 310. The edge portions are peripheral portions of the films 308 and 310 that surrounds the array of antennas pixels when the edge portions are attached to each other. The films may be glued or thermally sealed to each other at the edge portions although other possibilities for attaching the films to each other are conceivable. Note that the sizes of the antenna pixels and the flexible films are selected illustrative purposes and are not to scale.

Preferably, the antenna pixels 106 in the embodiment illustrated in FIG. 4A, are made from a two-dimensional material as described above, e.g. with reference to FIG. 2A-B. A two-dimensional material is bendable and optically transparent, see also FIG. 4B. Thus, the terahertz biometric imaging package 300 is both flexible, or bendable, and optically transparent. Further, the thickness of the terahertz biometric imaging package 300 is mainly the thickness of the films 310 and 308, since the two-dimensional material is extremely thin. A total thickness of the terahertz biometric imaging package 300 can be made less than 100 μm. Thereby, the thickness of the terahertz biometric imaging package 300 advantageously provides for mounting in many different locations without adding significant stack-up height. In addition, since it can be made optically transparent, it can be mounted almost anywhere without obstruction of visual appearance on the mounting surface. For example, the terahertz biometric imaging package 300 may be attached to the outer surface of a user device, e.g. on the display of a mobile device such as a mobile phone, tablet, or on a smart card, etc.

FIG. 4B conceptually illustrates a perspective view of the terahertz biometric imaging package 300 shown in FIG. 4A. FIG. 4B conceptually illustrates that the terahertz biometric imaging package 300 is bendable which further provides for simple mounting on surfaces of various shapes.

The bending capability of the terahertz biometric imaging package 300 depends primarily on the flexibility of the substrate where the two-dimensional material for forming the antenna pixels is deposited. The bend angle may be even as large as about 90 degrees or more for some substrates.

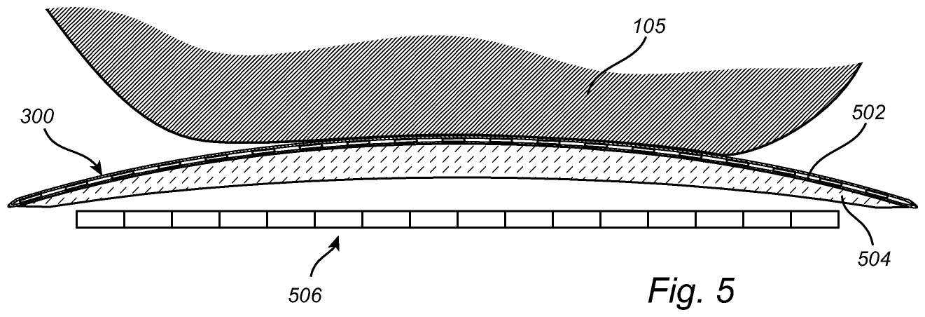

FIG. 5 conceptually illustrates a possible implementation of the terahertz biometric imaging package 300. Here, the terahertz biometric imaging package 300 being formed from two films 308 and 310 that enclose the two-dimensional antenna pixels 106 therebetween, is attached to the outer surface 502 of a display, e.g. on a cover glass 504. Since the terahertz biometric imaging package 300 is optically transparent, it will not prevent the user from seeing what is being shown on the display, as produced by e.g. an display device 506 such as LED, LCD, OLED, or similar arranged under the cover glass 504 of the display. Further, since the terahertz biometric imaging package 300 is flexible or bendable, it can be formed to be conformally shaped with the outer surface 502 of the cover glass 504.

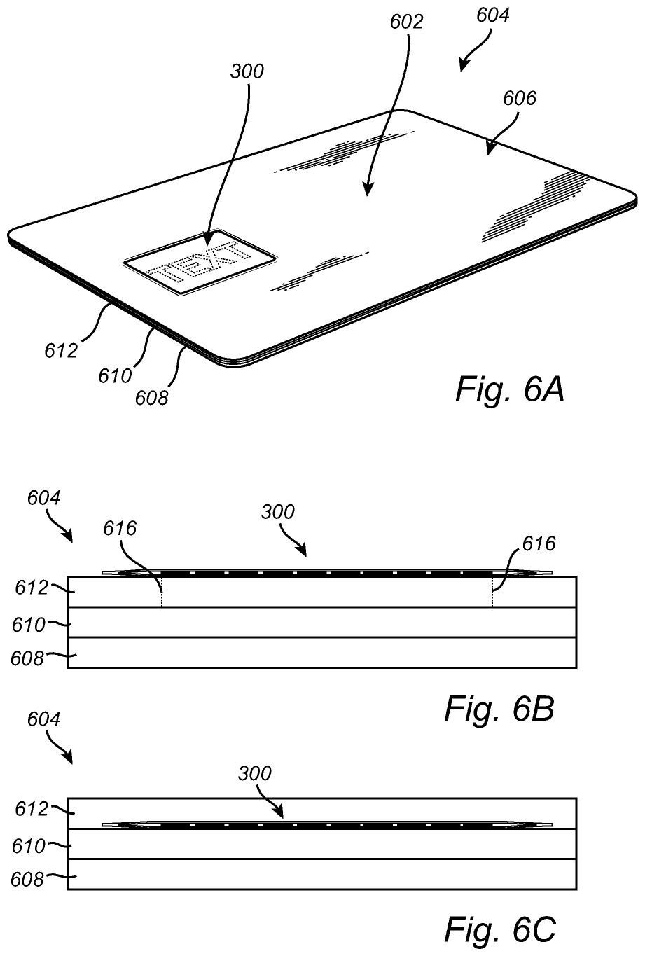

FIG. 6A conceptually illustrates a possible implementation of the terahertz biometric imaging package 300. Here, the terahertz biometric imaging package 300 being formed from two films 308 and 310 that enclose the two-dimensional antenna pixels 106 therebetween, is attached to the outer surface 602 of a smart card 604. Generally, the smartcard 604 comprises a bendable main body 606 made in a laminated structure comprising a plurality of layers 608, 610, 612. The transparent terahertz biometric imaging package 300 may be arranged on the outermost layer 612 layer of the smartcard 604, even on decoration and printing, see “text” in the layer 612 being visible through the transparent terahertz biometric imaging package 300.

The main body 606 is adapted to carry an electrical circuit external to the terahertz biometric imaging package 300. The layer 610 is an inlay layer which may comprise various electrically conductive traces acting as antennas and for connecting electronic components that may be included in the card 604. Layers 608 and 612 are outer layers protecting the inlay layer 610 and may include cosmetic decoration and printing as indicated by the printed “text” located under the transparent terahertz biometric imaging package 300. The layers 608, 610, 612 may be made of PVC and be laminated together. Due to the penetration properties of the terahertz radiation, the terahertz biometric imaging package 300 may be arranged between any two layers and still be able to capture an image of an object contacting the outer surface of the smartcard.

The bendable and transparent terahertz biometric imaging package 300 may equally well be arranged under the uppermost lamination layer 612 of the smart card 604 as on top of the layer 612. Regardless, the transparent terahertz biometric imaging package 300 advantageously does not obstruct the visual appearance of the smart card 604.

FIG. 6B is a cross-section conceptually illustrating the terahertz biometric imaging package 300 arranged on the top layer 612 of the smartcard 604, e.g. by gluing the package 300 to the top surface. Electrical connection leads 616 arranged through the top layer 612 and to the inlay layer 610 where electrically conductive lines are configured to electrically connect the terahertz biometric imaging package 300 to electrical circuitry external to the terahertz biometric imaging package 300.

FIG. 6C is a cross-section conceptually illustrating the terahertz biometric imaging package 300 arranged sandwiched between the inlay layer 610 and the top layer 612 of the smartcard 604. The provides for relatively straight forward electrically connecting the terahertz biometric imaging package 300 to electrical circuitry external to the terahertz biometric imaging package 300 via electrically conductive lines in the inlay layer 610. Further, the terahertz biometric imaging package 300 is advantageously fully integrated inside the smartcard 604 which provides protection for the terahertz biometric imaging package 300.

Some of the embodiments illustrated herein are directed to a passive sensor that does not require any assisting terahertz illumination of the object being imaged. In other embodiments, the terahertz biometric imaging package comprises a transmitter element arranged to emit terahertz radiation for illuminating the object. In such embodiments, the emitted terahertz radiation is reflected by the object, and subsequently detected by the image sensor.

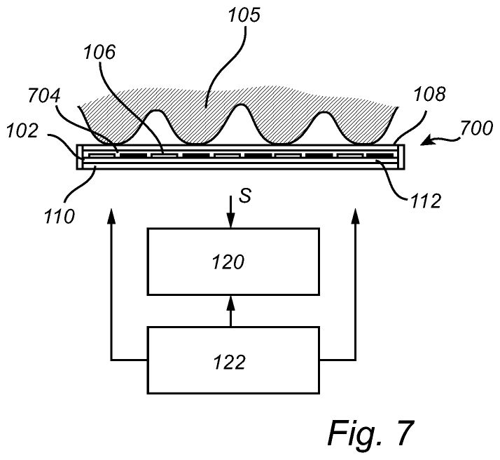

FIG. 7 illustrates a terahertz biometric imaging package 700 that comprises transmitter elements 702 arranged on the same substrate 112 as the array of antenna pixels. The antenna pixels and the transmitter elements are thus arranged side-by-side interleaved in the same array 704. As with the embodiment shown in FIG. 1, this embodiment also comprises the package top cover 108 and the package bottom part 110, here they enclose the array 704 of antenna pixels 106 and transmitter elements 702. The combined array 704 of antenna pixels 106 and transmitter elements 702 is applicable to the embodiments shown in each of the embodiments and implementations described herein.

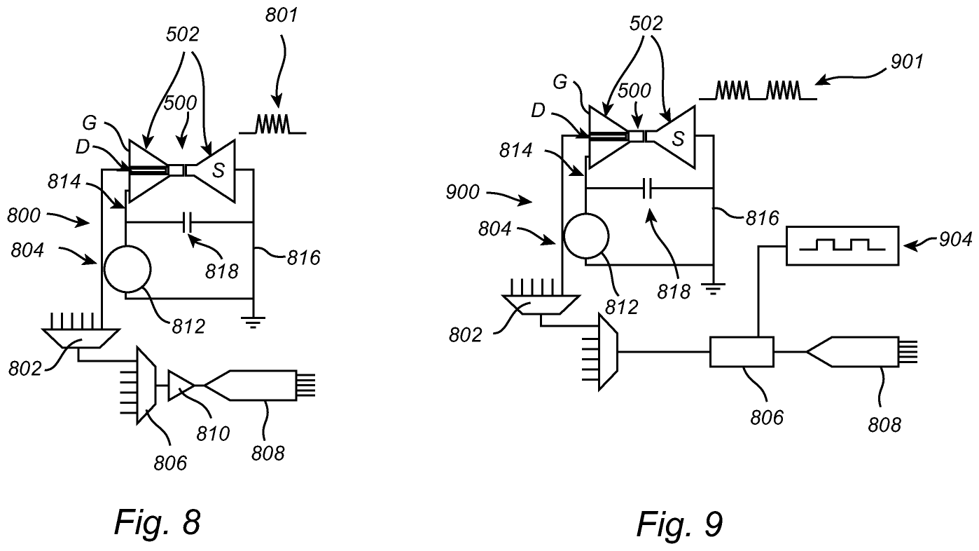

Turning to FIG. 8 which illustrates an example read-out circuit 800 for a power detector 500 configured to detect incoming terahertz radiation 801. The drain electrode D is connected to a multiplexer 802 via a read-out line 804, and a further multiplexer 806 may be connected in series with the first multiplexer 802 in order to handle signals from the rows and columns of power detectors in the array 104. The signals from the power detector 500 are low frequency or DC signals. The output of the multiplexer 806 is connected to an analog-to-digital converter 808 in series for sampling and converting the analog signals originating from the power detector 500 to a digital representation of e.g. the fingerprint pattern of a finger 105. In some implementations, an amplifier circuit 810 is inserted between the second multiplexer 806 and the ADC 808, although this is not strictly required.

A direct current source 812 is connected through lines 814 and 816 to the gate G and source S, respectively. The DC source 812 is arranged to feed the power detector 500 with a DC voltage. The gate G, and the source S, are connected through the capacitor 818, effectively providing a diode-connected transistor at high frequencies, i.e. the gate G and the source S are electrically shorted through the capacitor 818 at sufficiently high frequencies as tailored by the capacitor, preferably at frequencies exceeding the lower range of the terahertz frequencies desirable to detect for imaging.

Generally, the incoming terahertz radiation is detected through half-wave rectification and low-pass filtering. More specifically, when radiation 801 impinges on the gate G and the source S serving as antennas 502 of the power detector 500, the electrical potential of the gate G and the source S is modulated at the frequency of the incoming terahertz radiation 801, whereby the DC voltage feed is passed to the drain D. However, due to the diode-tied transistor configuration, the output at the drain D, is a half-wave rectified signal. This half-wave rectified signal is filtered through e.g. capacitors and/or inductive components (not shown) such as coils, to thereby provide a DC or low-frequency sensing signal to the multiplexor 802. For example, a capacitor may be inserted in parallel across the drain D, and ground, and/or inductive components may be connected in series with the drain D of the power detector 500. Accordingly, the power detector 500 operates as a rectifying transistor and as an antenna.

FIG. 9 illustrates another example read-out circuitry 900 in which the output of the second multiplexer 806 is connected to a lock-in amplifier 902. The lock-in amplifier 902 is configured to receive a reference signal from the transmitter element 904. The transmitter element 904 is adapted to generate the terahertz radiation that is reflected by the object, e.g. resulting in the radiation 901 to be detected. The generated terahertz radiation is pulsed at a set frequency. The set frequency is used as a reference for the lock-in amplifier which in this way selectively measures the terahertz radiation transmitted from the object by tuning at the same frequency as the pulsation frequency of the terahertz radiation generated by the transmitter element 904.

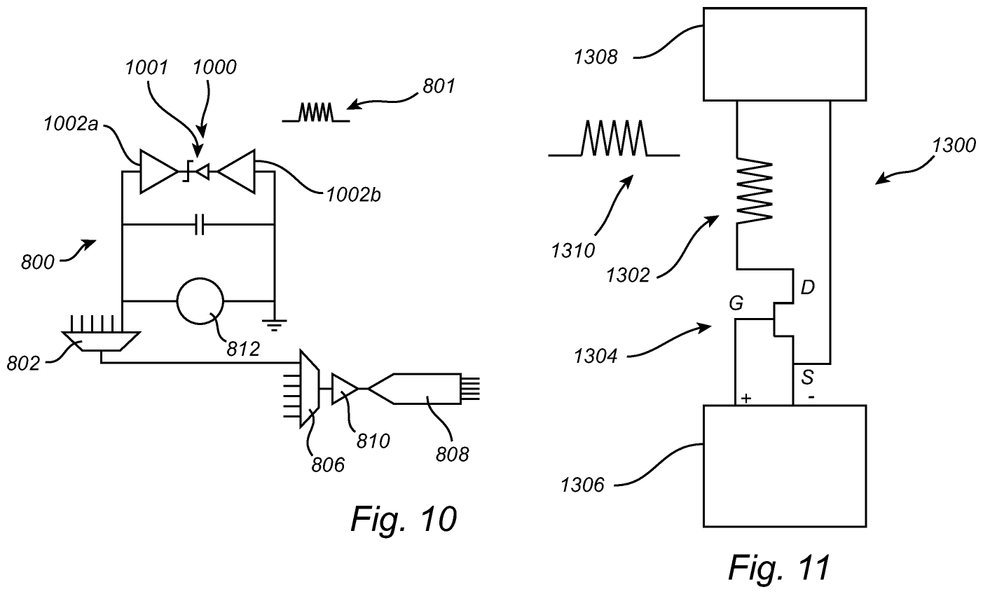

FIG. 10 illustrates another possible implementation of the inventive concept in which a power detector 1000 in the form of a dipole antenna sensor with a rectifying diode 1001 connected between the receiver antennas 1002a-b. The read-out circuit 800 is in this implementation the same as the one described with reference to FIG. 8.

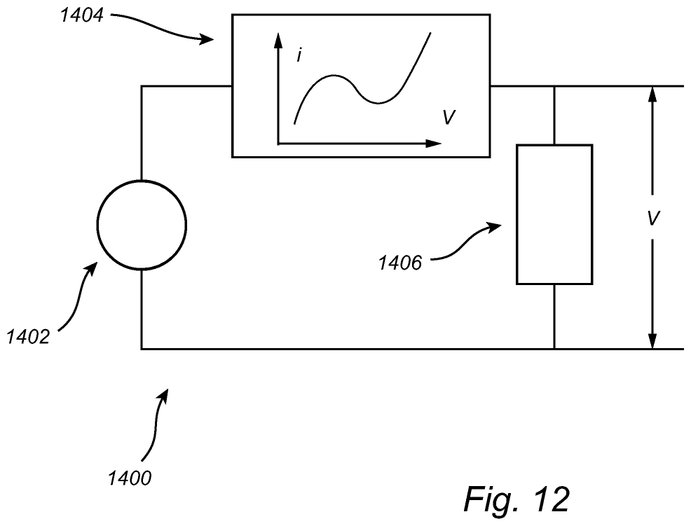

Various types or transmitter elements are applicable and FIG. 11 and FIG. 12 conceptually illustrate to conceivable transmitter elements.

FIG. 11 conceptually illustrates an example transmitter element in the form of a black body transmitter that may be implemented as a filament film. The example black body transmitter 1300 comprises a resistive element 1302 and a transistor 1304. The source of the transistor is connected to a controllable pulse generator 1306 which is also connected to the gate of the transistor 1304. A power supply 1308 is connected to provide a current to the source. As the controllable pulse generator provides a pulse to the gate, the resistance through the transistor decreases whereby the current from the power supply 1308 passes through the transistor and to the resistive element 1302, from which terahertz radiation 1310 is produced.

FIG. 12 conceptually illustrates an example transmitter element in the form of a negative resistance oscillator 1400 comprising a power supply 1402 connected on a negative resistance device such as e.g. a tunnel diode or IMPATT diode, and in parallel with a resonant circuit 1406. The output V is the source for the terahertz radiation.

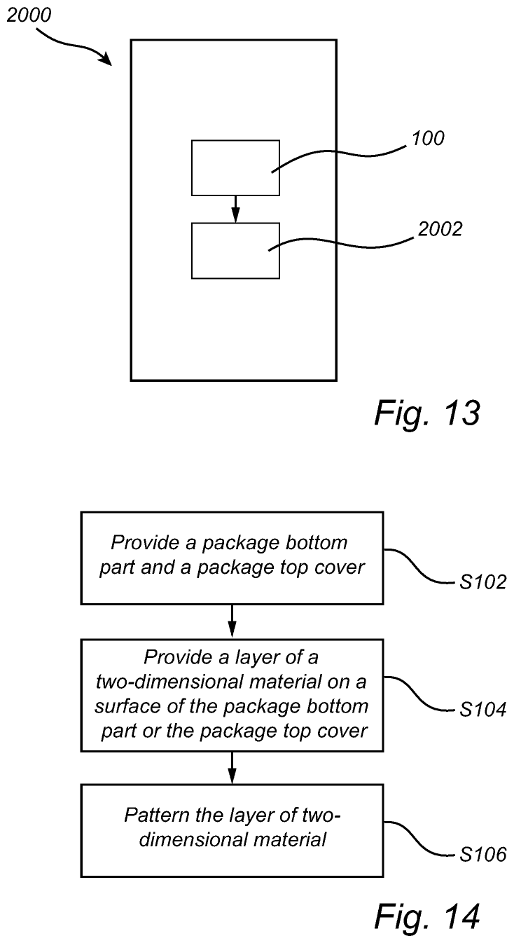

FIG. 13 is a schematic box diagram of an electronic device according to embodiments of the invention. The electronic device 2000 comprises a terahertz biometric imaging package 100 Furthermore, the electronic device 2000 comprises processing circuitry such as control unit 2002. The control unit 2002 may be stand-alone control unit of the electronic device 2002, e.g. a device controller. Alternatively, the control unit 202 may be comprised in the terahertz biometric imaging package 100.

FIG. 14 is a flow-chart of method steps for manufacturing an image sensor for a terahertz biometric imaging package. The method comprises a step S102 of providing a package bottom part and a package top cover for a terahertz biometric imaging package. Step S104 includes providing a layer of a two-dimensional material on a surface of the package bottom part or on the package top cover. In subsequent step S106 patterning the layer of two-dimensional material to form an array of antenna pixels configured to detect terahertz radiation.

In embodiments, the package bottom part and the package top cover may be flexible and transparent films, the method comprising laminating the flexible and transparent films to each other such that the array of antenna pixels is enclosed therebetween.

Note that the sizes of the antenna pixels, flexible films, package top cover, package bottom part, transmitter element, and other components of the package selected for clarity and are not necessarily to scale.

A control unit may include a microprocessor, microcontroller, programmable digital signal processor or another programmable device. The control unit may also, or instead, include an application specific integrated circuit, a programmable gate array or programmable array logic, a programmable logic device, or a digital signal processor. Where the control unit includes a programmable device such as the microprocessor, microcontroller or programmable digital signal processor mentioned above, the processor may further include computer executable code that controls operation of the programmable device. It should be understood that all or some parts of the functionality provided by means of the control unit (or generally discussed as “processing circuitry”) may be at least partly integrated with the biometric imaging package.

Even though the invention has been described with reference to specific exemplifying embodiments thereof, many different alterations, modifications and the like will become apparent for those skilled in the art. Also, it should be noted that parts of the biometric imaging package may be omitted, interchanged or arranged in various ways, the imaging device yet being able to perform the functionality of the present invention.

Additionally, variations to the disclosed embodiments can be understood and effected by the skilled person in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage.

1. A terahertz biometric imaging package comprising:
an image sensor comprising an antenna pixel array arranged to detect terahertz radiation transmitted from an object for capturing an image, each antenna pixel comprising a power detector including an antenna structure for sensing terahertz radiation and a frequency converting element configured to convert the sensed terahertz radiation to a sensing signal having a lower frequency than the frequency of the sensed terahertz radiation,
a package top cover arranged to cover the antenna pixel array, wherein the image sensor is configured to capture a terahertz image of an object located on an opposite side of the package top cover, wherein the package top cover is a flexible transparent film,
a package bottom part arranged on the other side of the antenna pixel array opposite from the package top cover, wherein the antenna pixel array is encapsulated between the package top cover and the package bottom part, wherein the package bottom part is a flexible transparent film,
wherein one of the package bottom part and the package bottom part is configured as a substrate for the array of antenna pixels.
wherein the package top cover and the package bottom part are attached to each other with the array of antenna pixels in-between.

2. The terahertz biometric imaging package according to claim 1, wherein the sensing signal is extractable from the antenna pixels of the image sensor for redirecting directly to an analogue to digital converter of a read-out circuit for sampling and converting the sensing signals to a digital representation of the object.

3. The terahertz biometric imaging package according to claim 1, wherein the power detector comprises at least one on-chip transistor structure connected to the antenna structure of the antenna pixel.

4. The terahertz biometric imaging package according to claim 3, wherein the transistor structure and the antenna structure are made in a single component.

5. The terahertz biometric imaging package according to claim 1, wherein the power detector is made from a two-dimensional material.

6. The terahertz biometric imaging package according to claim 1, wherein the array of antenna pixels is manufactured on the package bottom part.

7. The terahertz biometric imaging package according to claim 1, wherein the array of antenna pixels is manufactured on the package top cover.

8. The terahertz biometric imaging package according to claim 1, comprising a transmitter element arranged to emit terahertz radiation for illuminating the object.

9. The terahertz biometric imaging package according to claim 1, wherein the transmitter element and the array of antenna pixels are arranged on the same substrate.

10. The terahertz biometric imaging package according to claim 9, wherein an array of transmitter elements is arranged interleaved with the array of antenna pixels on the same substrate surface.

11. The terahertz biometric imaging package according to claim 8, wherein the transmitter element comprises a thermal emitting filament.

12. The terahertz biometric imaging package according to claim 8, wherein the transmitter element comprises at least one non-linear device diode or a transistor.

13. The terahertz biometric imaging package according to claim 1, wherein the image sensor comprises a substrate supporting the antenna pixel array, wherein the substrate is made from a flexible material.

14. The terahertz biometric imaging package according to claim 1, wherein the antenna pixel array is a two-dimensional array of antenna pixels.

15. The terahertz biometric imaging package according to claim 14, configured to be directly attached to a surface of a user device.

16. The terahertz biometric imaging package according to claim 15, wherein the surface is an outer surface of a display cover glass.

17. The terahertz biometric imaging package according to claim 1, wherein the image sensor is operative to detect terahertz radiation in a frequency range excluding the range of visible light.

18. The terahertz biometric imaging package according to claim 1, wherein the image sensor is operative in the frequency range 10 GHz to 100.

19. An electronic device comprising:
the terahertz biometric imaging package according to claim 1, and
processing circuitry configured to:
receive a signal from the terahertz biometric imaging arrangement indicative of a biometric object touching the transparent display panel,
perform a biometric authentication procedure based on the detected fingerprint.

20. (canceled)

21. A method of manufacturing an image sensor for a terahertz biometric imaging package, the method comprising:
providing a package bottom part and a package top cover for a terahertz biometric imaging package, wherein the package bottom part and the package top cover are flexible and transparent films,
providing a layer of a two-dimensional material on a surface of the package bottom part or the package top cover;
patterning the layer of two-dimensional material to form an array of antenna pixels each comprising a power detector including an antenna structure for sensing terahertz radiation and a frequency converting element configured to convert the sensed terahertz radiation to a sensing signal having a lower frequency than the frequency of the sensed terahertz, and
laminating the flexible and transparent films to each other such that the array of antenna pixels is enclosed therebetween.
